(19) United States
(12) Patent Application Publication
(10) Pub. No.: US 2019/0076141 A1
(43) Pub. Date: Mar. 14, 2019

(54) KNOTTING TUBE

(71) Applicant: Children's Medical Center Corporation, Boston, MA (US)

(72) Inventor: Kaifeng Liu, Boston, MA (US)

(21) Appl. No.: 16/084,425

(22) PCT Filed: Mar. 17, 2017
(86) PCT No.: PCT/US17/22899

§ 371 Date: Sep. 12, 2018

Related U.S. Application Data
(60) Provisional application No. 62/309,569, filed on Mar. 17, 2016.

(51) Int. Cl. A61B 17/04

(52) U.S. Cl. CPC A61B 17/0487; A61B 2017/0409; A61B 2017/0454

(57) ABSTRACT

A suture lock device is provided that includes a tube and a plurality of protrusions. The tube has a passageway formed therethrough and the protrusions extend from at least a portion of an inner surface of the tube to pierce a suture. The tube receives both ends of the suture through the passageway and is then moved to abut a surgical site. The protrusions are deformable in one direction to lock the suture in place and maintain the tube to abut the surgical site.

### RELATED APPLICATIONS

This application claims the benefit of priority under 35 U.S.C. § 119(e) to U.S. Provisional Application No. 62/309,569, filed Mar. 17, 2016, which is incorporated herein by reference in its entirety.

### TECHNICAL FIELD

The present disclosure relates generally to a knotting tube, and more particularly, to a knotting tube used as a suture lock that receives both suture ends and locks the suture against a surgical site.

### BACKGROUND

Generally, sutures are used in medical procedures to close surgical wounds, hold tissues together, or the like. Conventionally, a suture looped through a needle is passed in and out of a surgical site to close the wound. Such a procedure requires accurate manipulation and requires the application of sufficient tension to secure the suture against the surgical site. For example, as seen in FIG. 1 of the prior art, the free ends 110 of the suture are tied together with a knot 105 to hold the suture against the surgical site and to hold the closure together.

However, human error may cause insufficient tension to be applied to the knot, thus causing less accuracy in the closing of a wound. The traditional knot tying technique is time consuming and requires extensive training for medical professionals to be capable of accurately securing the suture to a surgical site. Further, during endoscopic or laparoscopic surgeries, the knot tying procedure of the suture is executed remotely from the surgical site and the knot is manipulated using a medical instruction to slide the knot toward the surgical site. Such a manipulation and remote execution further increases the risk of human error.

Various techniques have been researched for improving the accuracy of these medical procedures, such as for example, a clip applied to the ends of the suture. However, such a technique is limited in application due to the size thereof and difficulty of manipulation near the tissue. The technique is thus not capable of being applied against a surgical site with sufficient tension to accurately close a wound. The length and rigid property of such a clip may also cause potential injury to neighboring tissues. Accordingly, to improve feasibility of medical procedures it is necessary to provide a device that decreases the training time of traditional knot tying sutures while increasing the accuracy of tension against a surgical site.

### SUMMARY

We now provide a knotting tube used as a suture lock that receives both suture ends and locks the suture against a surgical site.

According to one aspect, a suture lock is provided that comprises a tube having a passageway formed therethrough and a plurality of protrusions that extend from at least a portion of an inner surface of the tube to pierce a suture. In particular, the tube receives both ends of the suture through the passageway and is moved to abut a surgical site. The protrusions are deformable in one direction to thus lock the suture and maintain the tube abutting the surgical site. Additionally, the protrusions may be formed at varied lengths, at an angle less than 90 degrees, and in various shapes including a barb, valve, thorn, hook, and the like.

According to another aspect, a longitudinal side of the tube may be open to receive the suture through a side opening. In addition, a handle may be disposed at an opposite side of the side opening. The inner surface of the tube may have a rough topography to grasp and lock the suture against the surgical site. The suture lock may also be made of a biomaterial.

In another exemplary embodiment, two tubes may be disposed adjacently to receive multiple sutures. A guide may further be provided to pull both ends of the sutures through each tube passageway. The tube of each suture may have a varied cross-sectional shape such as a circular, rectangular, and triangular cross-sectional shape. The lock suture itself may also be formed in a bead shape having protrusions formed on an inner surface thereof to pierce the suture.

According to another aspect, a method of locking a suture against tissue is provided. In particular, the method may include pulling the suture through the tissue by piercing a needled engaged with the suture through the tissue and then passing the ends of the suture through a tube of a suture lock. The suture lock may then be pushed against the tissue to engage the suture in the tube with protrusions extending from at least a portion of an inner surface of the tube to pierce the suture and lock the suture against the tissue.

Notably, the present invention is not limited to the combination of the knotting tube elements as listed above and may be assembled in any combination of the elements as described herein.

Other aspects of the invention are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates a conventional tying of suture according to the prior art;

FIG. 2A illustrates the knotting tube according to an exemplary embodiment of the present disclosure;

FIGS. 2B-2C illustrates the protrusions of the knotting tube according to an exemplary embodiment of the present disclosure;

FIGS. 3A-3B illustrate a guiding loop passed through the knotting tube according to an exemplary embodiment of the present disclosure;

FIGS. 4A-4B illustrate the threads guided through the guiding loop according to an exemplary embodiment of the present disclosure;

FIG. 5 illustrates the pulling of the threads through the knotting tube according to an exemplary embodiment of the present disclosure;

FIGS. 6A-6C illustrate a detailed view of the threads pulled through the knotting tube according to an exemplary embodiment of the present disclosure;

FIG. 7 illustrates two knotting tubes used in conjunction according to an exemplary embodiment of the present disclosure;

FIG. 8 illustrates the knotting tube pushed toward a surgical site according to an exemplary embodiment of the present disclosure;

FIGS. 9A-9C illustrate the penetration of the protrusions into the threads according to an exemplary embodiment of the present disclosure;

FIG. 10 illustrates the knotting tube locked in place against a surgical site according to an exemplary embodiment of the present disclosure; and

FIGS. 11A-11B illustrate a suture lock having a handle and a side opening according to another exemplary embodiment of the present disclosure.

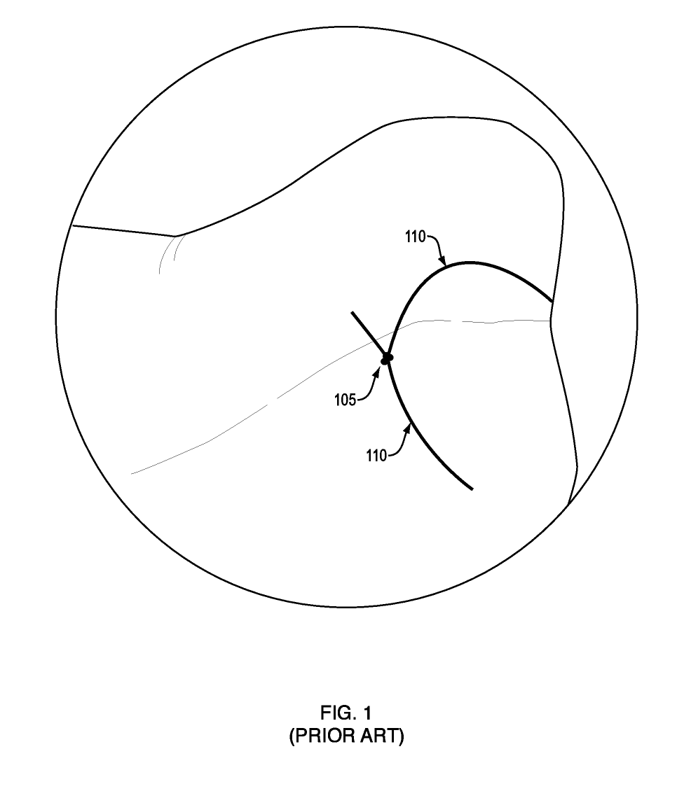

### DETAILED DESCRIPTION

The presently disclosed subject matter will be described more fully hereinafter with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not to be limited to the specific embodiments disclosed and that modifications and other exemplary embodiments are intended to be included within the scope of the appended claims.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In one aspect, the present disclosure provides a knotting tube that is used as a suture lock having a tube and protrusions formed as one integrated component. The knotting tube allows for a simplified manner of securing a suture against a surgical site during a medical procedure. That is, the knotting tube omits the need for a knot to be tied to lock the suture against the surgical site which requires accurate technique and extensive training.

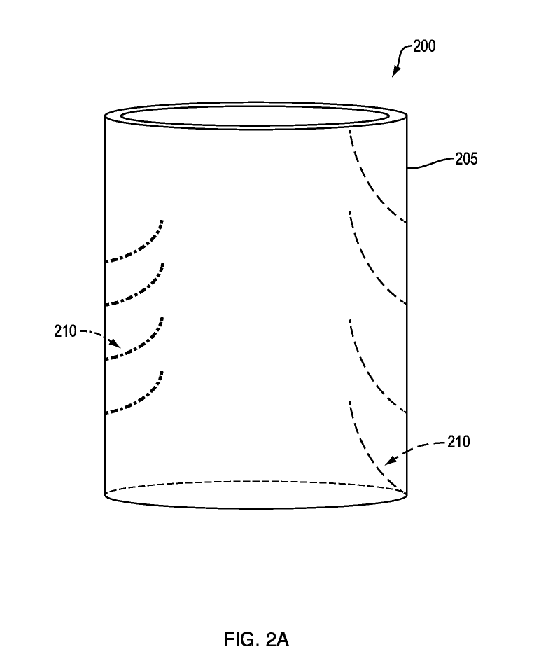

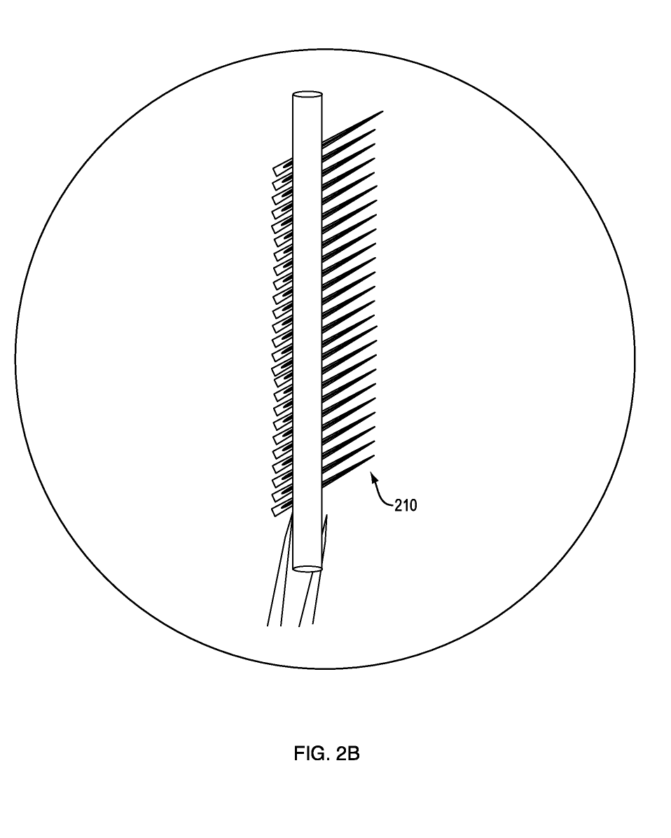

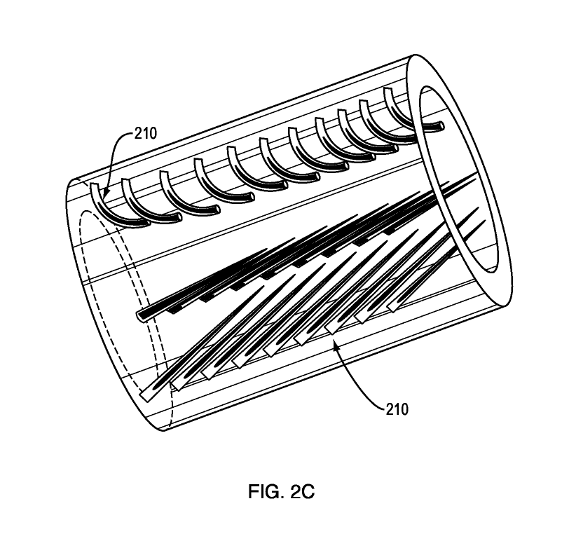

FIGS. 2A-2C illustrate a view of the suture lock according to an exemplary embodiment of the present disclosure. In particular, as seen in FIG. 2A, the suture lock 200 includes a tube 205 having a passageway formed therethough. The tube in FIG. 2A is shown to be a hollow cylindrical tube, but the disclosure is not limited thereto. For example, the cross-sectional shape of the tube may be circular, rectangular, triangular, or the like. The tube 205 may also vary in length. Further, the suture lock 200 includes a plurality of protrusions 210 that extend from at least a portion of an inner surface of the tube 205 to then pierce a suture (e.g., a thread) passed through the tube 205. The protrusions 210 may be formed at merely a portion of the tube 205 to facilitate the cutting of the tube without requiring the removal of the suture from the tube 205. Alternatively, the protrusions 210 may be formed along the entire inner surface of the tube 205.

Further, FIG. 2B shows a detailed view of the protrusions 210 which are formed to allow the suture to pass in one direction and pierce the suture in an opposite direction to thus lock the suture in place. The protrusions 210 may be formed in varied shapes such as a barb, valve, thorn, hook, and the like. Additionally, the protrusions 210 are not limited to a particular length and are capable of being formed at varied lengths while formed at an angle less than 90 degrees. The particular angle of less than 90 degrees thus allows the protrusions 210 to pierce or penetrate into the suture. FIG. 2C illustrates the various shapes of the protrusions 210. For example, FIG. 2C shows that the protrusions 210 may be needle shaped and rigid or alternatively, may be formed as soft protrusions that are bendable as the suture passes through the tube. The inner surface may also be formed with a rough topography. The rough topography may have a smooth surface in the direction in which the suture is pulled through the passageway of the tube and a rough surface in the opposite direction to thus hold the suture in place by friction between the surfaces.

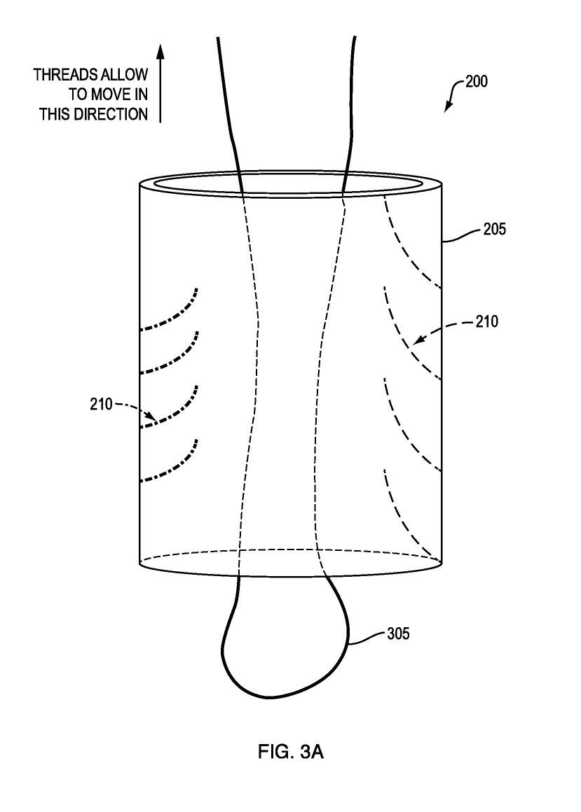

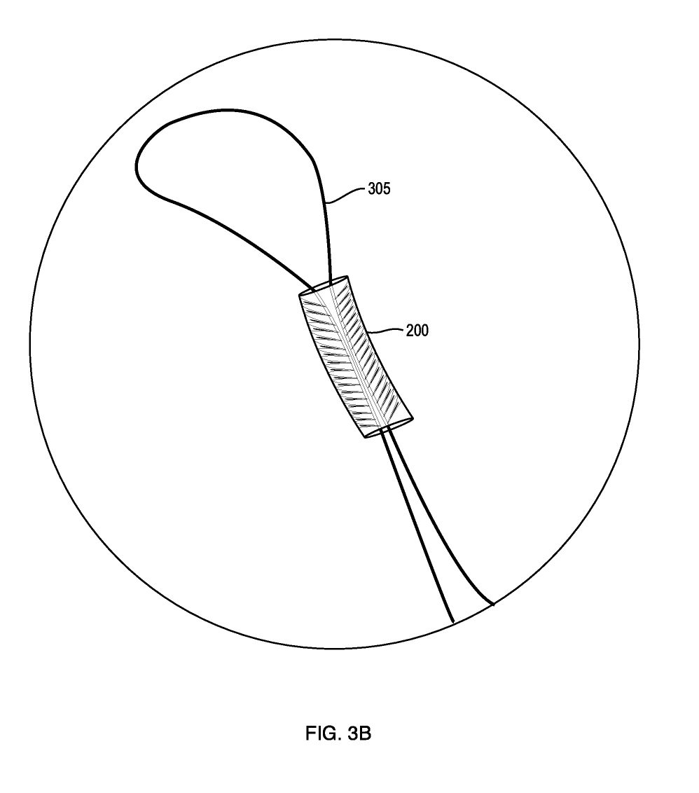

According to one exemplary embodiment, a guide may further be provided as shown in FIG. 3A-3B to pull both ends of the suture through the tube passageway. As shown in FIGS. 3A and 3B, the guide 305 may be pulled through the tube 205 to form a loop at one open end of the tube. The loop may then be used to pull the suture through the passageway. In particular, the guide 305 facilitates the insertion of the suture into the tube 205 given the narrow passageway that may be formed in the tube. The guide 305 may be made of a more rigid material to stably pull the suture through the tube 205. The arrow shown in FIG. 3A indicates the pulling direction of the guide 305 once the suture is wrapped around the loop of the guide 305.

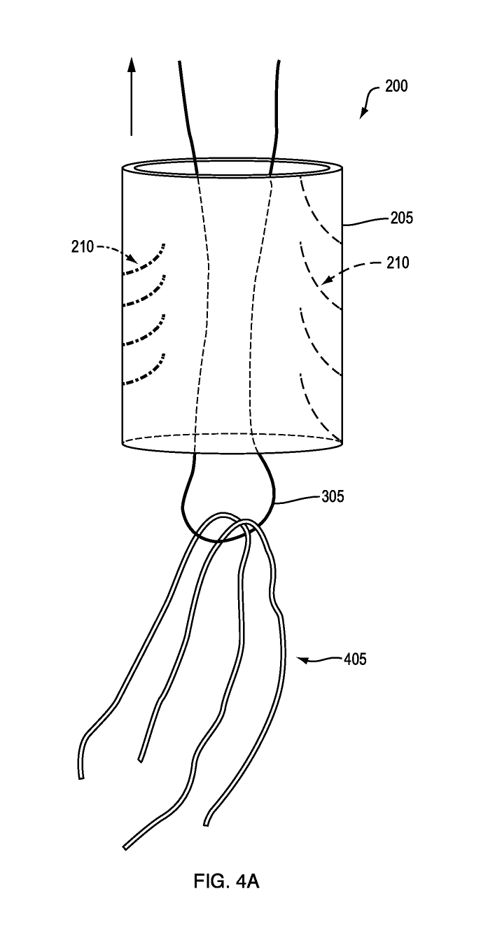

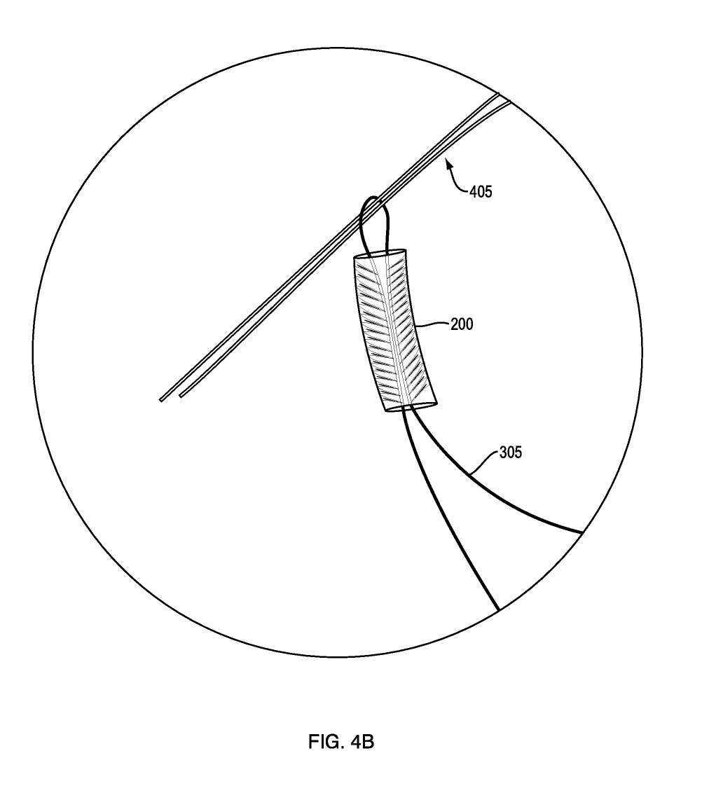

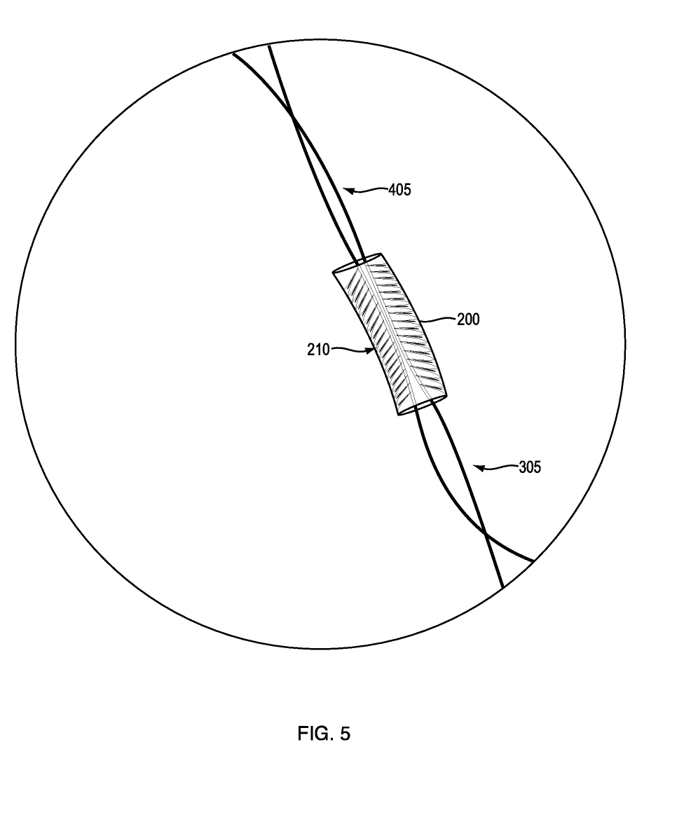

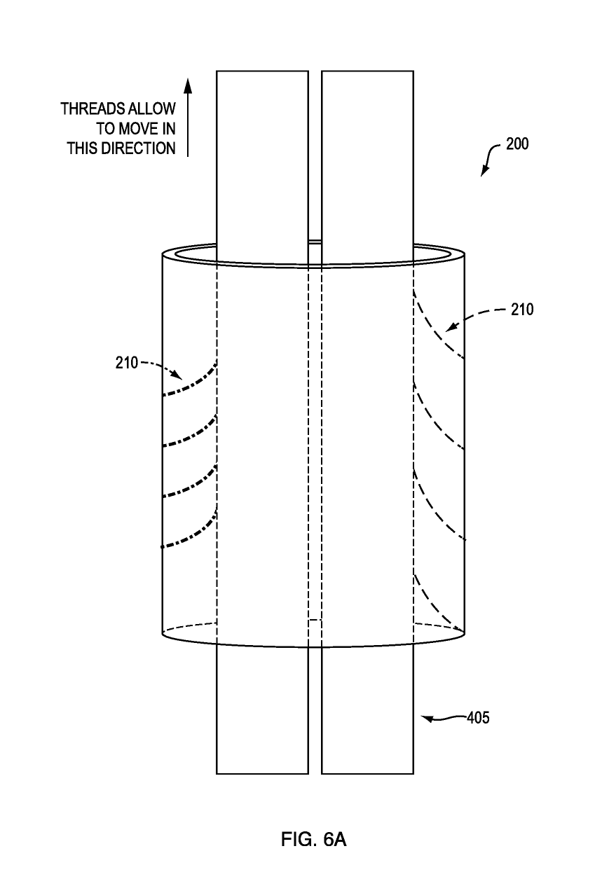

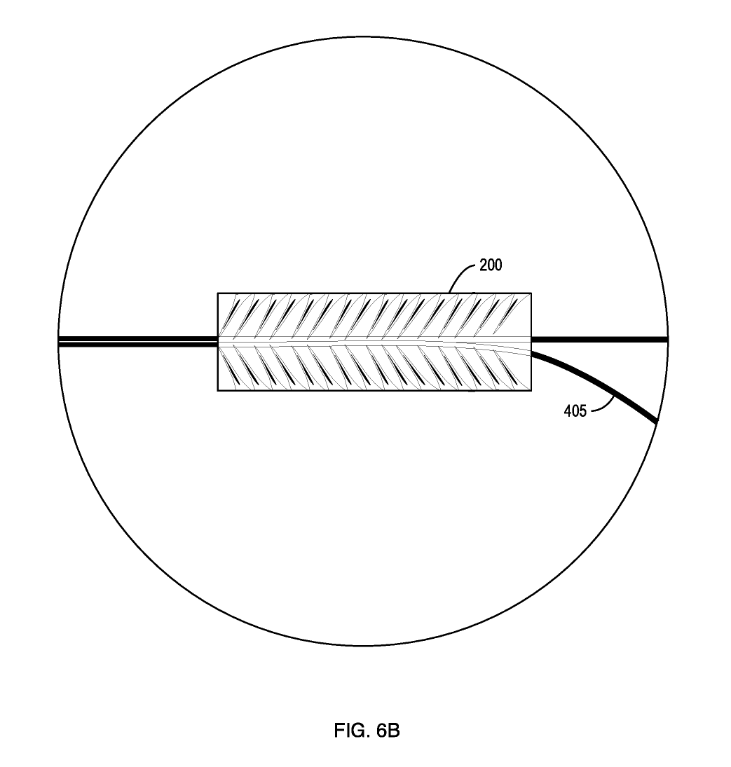

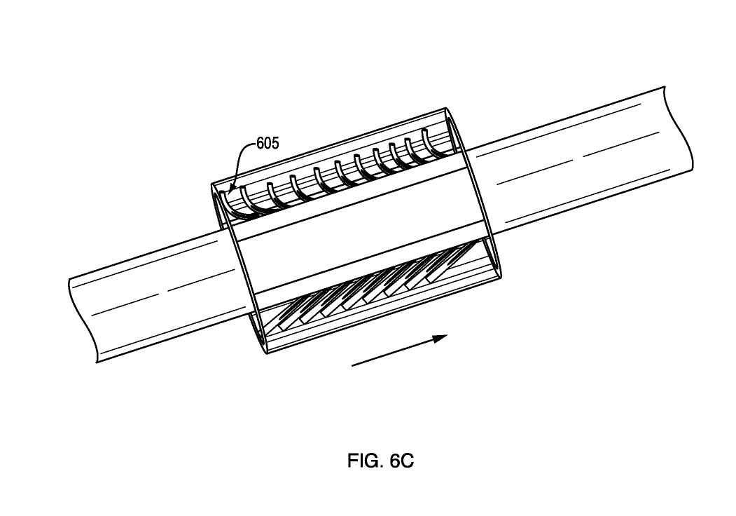

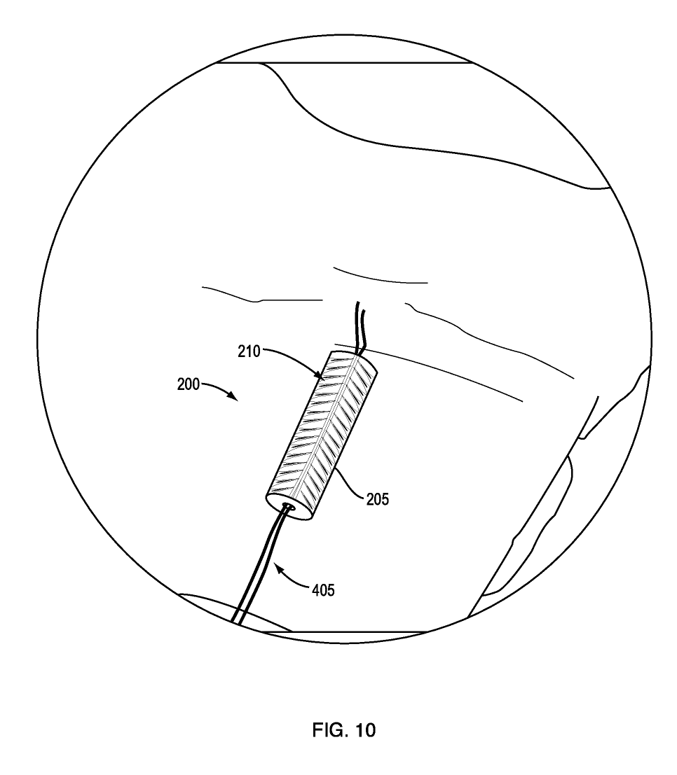

Particularly, once the suture has been pulled through a surgical site (e.g., tissue) by the piercing of a needled engaged with the suture through the tissue, the suture threads 405 may be looped around the guide 305 to pass through the passageway of the tube 205, as shown in FIGS. 4A-4B. The suture passes through the tube having the protrusions formed on the inner surface thereof and the pulling direction (indicated by an arrow in FIG. 4A) is along the protrusions, allowing the suture to continue passing through the passageway. FIG. 5 illustrates the pulling of the threads through the passageway of the tube along the protrusions. That is, the threads 405 are pulled along a smooth side of the protrusions. Further, FIGS. 6A-6C show the threads 405 having been pulled through the tube 205 and the guide having been removed. In particular, at this stage, a looped end of the suture is disposed through a surgical site or through tissue and the free ends of the suture have passed through the knotting tube, as shown later in more detail in FIG. 10. FIG. 6C illustrates the passageway and protrusions within the tube. The protrusions may bend along the threads as the suture is pulled in the direction shown in FIG. 6C to thus allow the suture to pass through the tube.

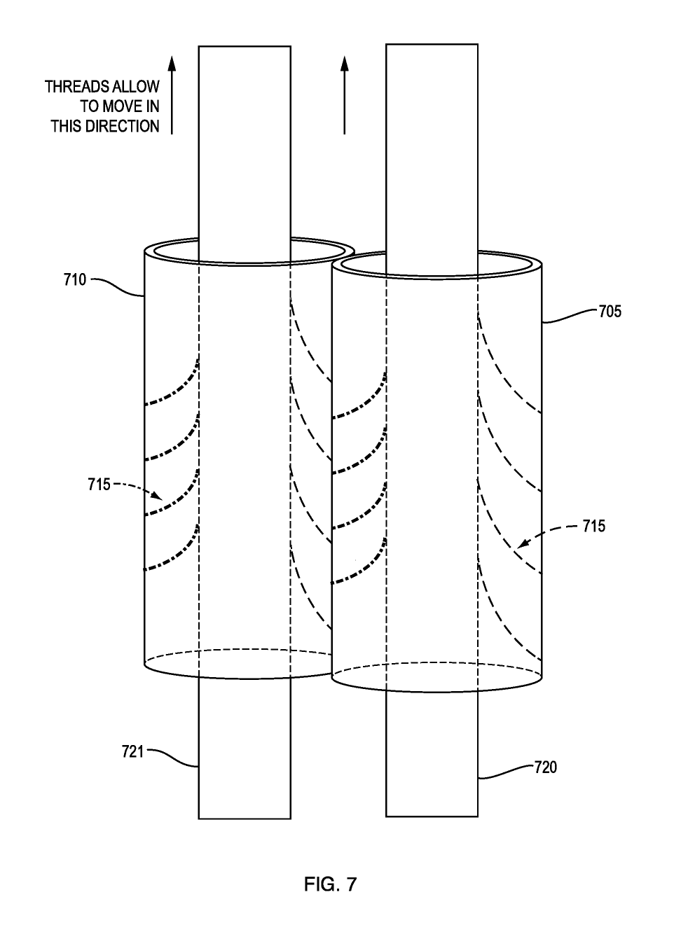

In another exemplary embodiment, multiple suture locks may be used in conjunction. For example, as shown in FIG. 7, a first tube 705 and a second tube 710 may be disposed adjacently and may each contain protrusions 715 that extend from an inner surface thereof to pierce the suture. The threads are capable of moving in the direction as shown by the arrow in FIG. 7. A first thread end 720 may move through the first tube 705 and a second thread end 721 may move through the second tube 710. Additionally, a guide may be used to guide the sutures through each of the first and second tubes. The use of multiple knotting tubes may be used during more complex medical procedures to facilitate the locking of a suture in multiple areas. Alternatively, multiple knotting tubes may be used for multiple surgical sites that are proximate to each other.

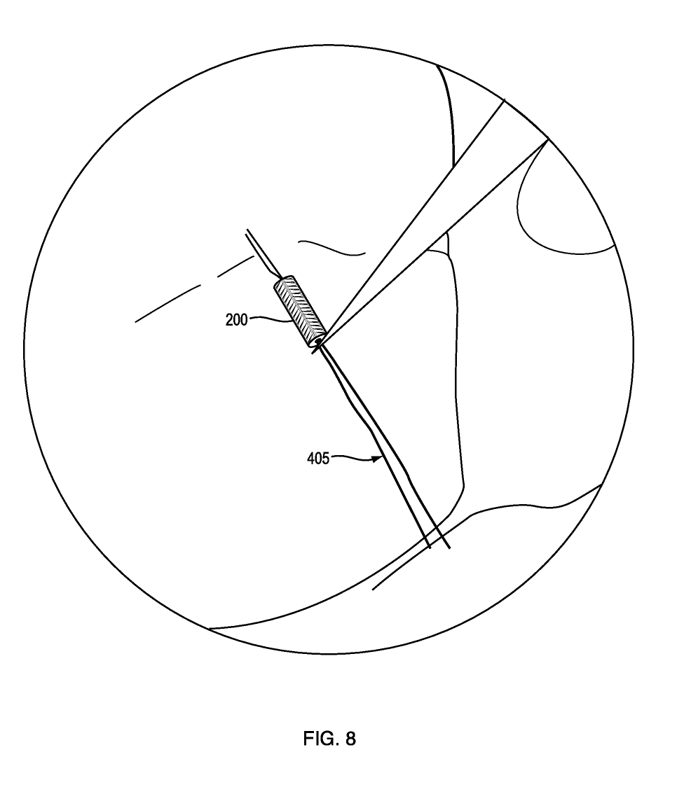

Furthermore, once the suture has been moved, pulled, or slid through the passageway of the tube, the knotting tube or suture lock may be pushed up against the surgical site. For example, FIG. 8 shows forceps or tweezers used to move the suture lock to abut the surgical site, however the disclosure is not limited thereto. Since the suture lock may vary in size to accommodate different medical procedures, medical instruments such as forceps may be used to grasp the tube of the suture lock or an end thereof to push the suture lock toward the surgical site. However, other devices may be used to facilitate the movement of the suture lock toward the suture lock. The suture lock may also be formed as a bead shape or the like to accommodate a minimal surgical site. That is, the suture lock is not limited in shape. Such a small shape may use the aid of a medical instrument to move the suture lock. However, the aid of medical instruments for the movement of the suture lock may also be omitted depending of the size of the suture lock.

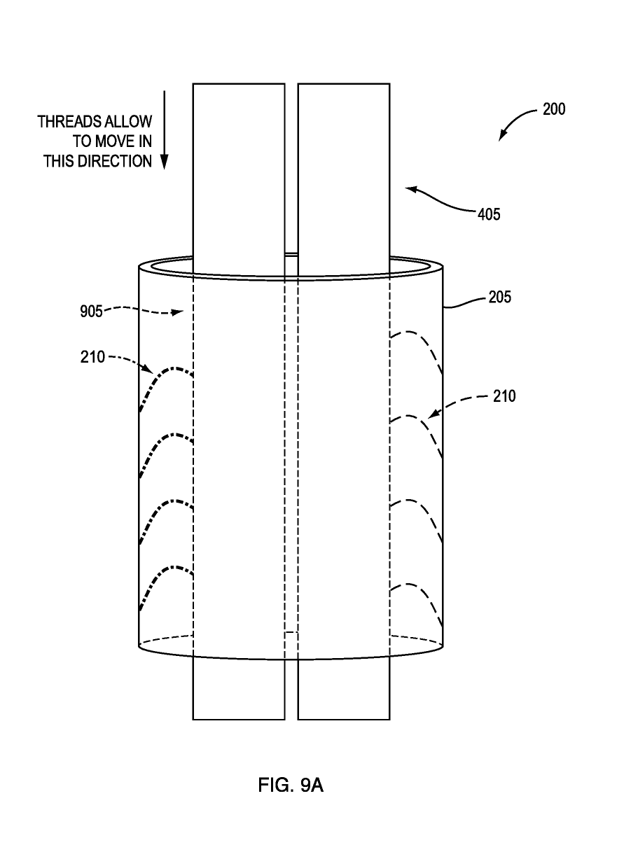

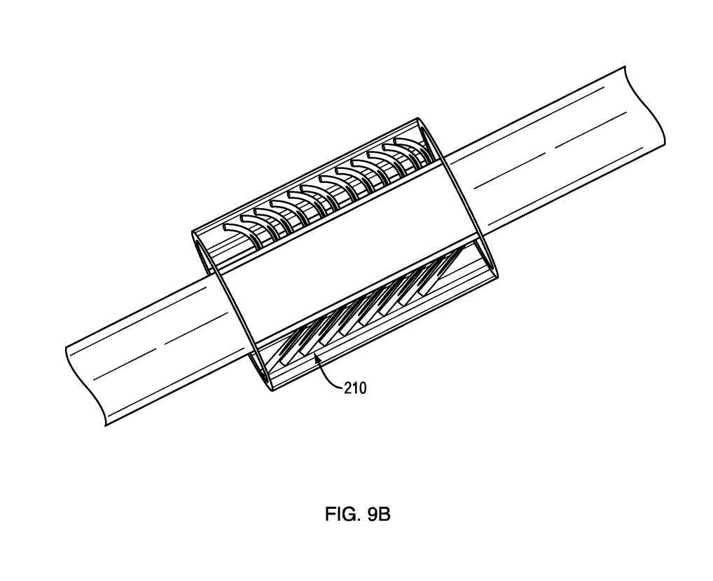

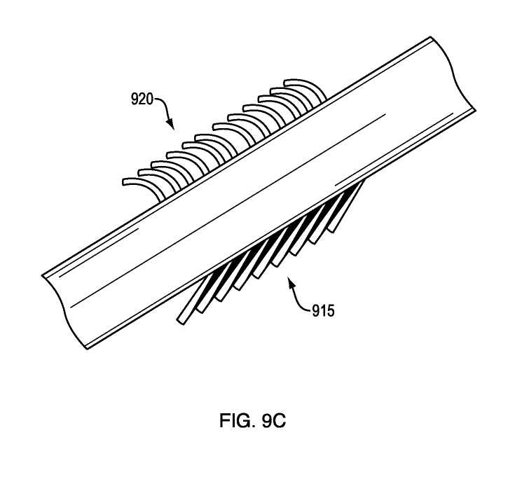

Moreover, the suture threads are then blocked from moving in a backward direction (e.g., an opposite direction to the entry into the passageway) to thus maintain the position of the suture lock against the surgical site. FIGS. 9A-9C illustrate the penetration of the protrusions 210 into the threads 405. The arrow shown in FIG. 9A indicates the direction in which the suture is blocked. Further, as shown in FIG. 9A, the piercing point 905 of the protrusion grasps the threads 405 to thus lock the suture in position within the tube 205. Accordingly, a knot tying step may be omitted while maintaining a strong and abutting connection between the suture and a surgical site. That is, the suture lock allows sufficient tension to be applied against the surgical site.

FIG. 9C details the penetration of a tip of the suture into the thread with the tube removed. When the protrusions 210 are formed as a rigid shape 915, the tip of the protrusion pierces into the thread. Alternatively, when the protrusions 210 are formed as a soft shape 920, the tip of the protrusion bends to create friction against the suture thus prevent the threads from moving out of the passageway of the tube. In another exemplary embodiment, the suture may be locked in place based on van der Waals forces or friction generated between the protrusion tip and the surface of the suture. That is, the van der Waals forces provide the required strength (e.g., adhesion forces) to hold the suture in place and lock the suture in a one-way direction. Furthermore, FIG. 10 illustrates the suture lock abutting a surgical site to complete a medical procedure without requiring additional tying of the suture. The suture lock may also be made of a biomaterial such that cell growth may occur over the tube to absorb the tube into the skin.

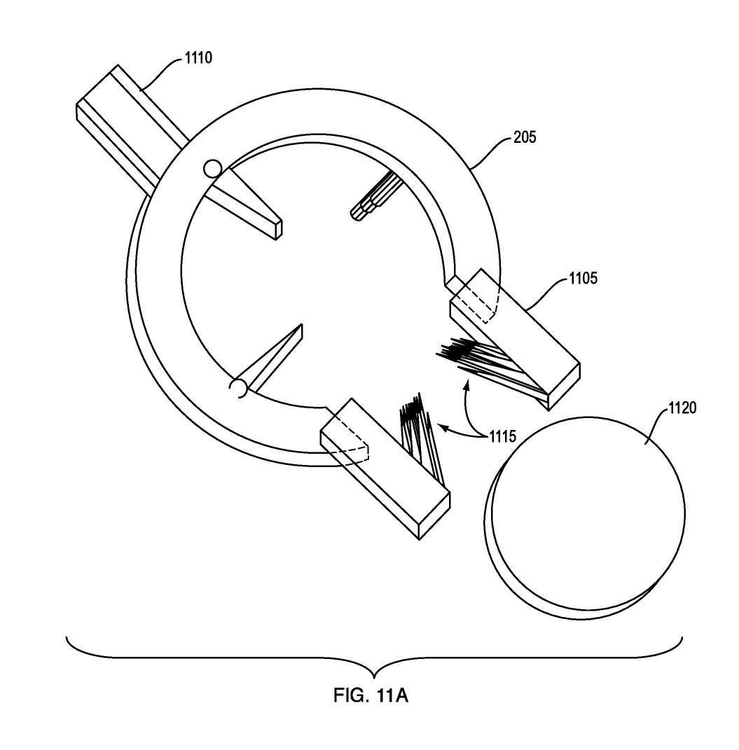

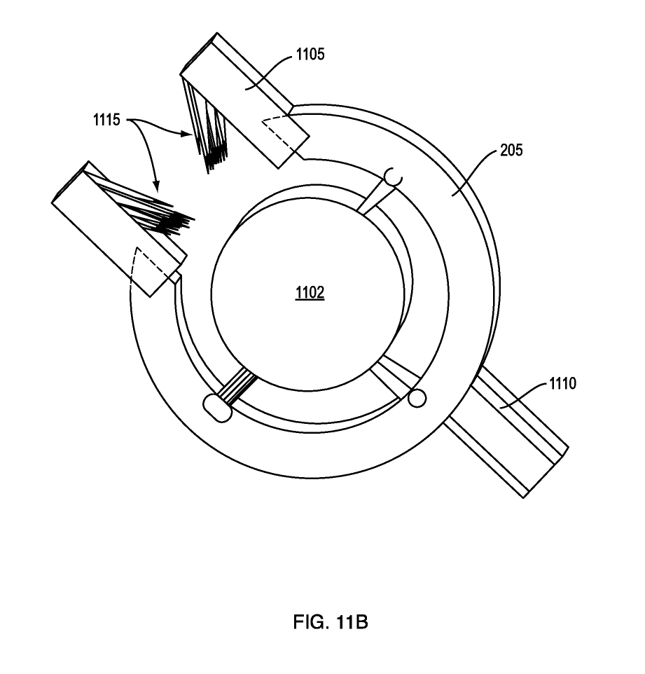

In another aspect of the present disclosure, the suture lock may be formed with an opening in a longitudinal side thereof. As shown in FIG. 11A, the side opening 1105 of the suture lock may include protrusions 1115. The suture 1120 may enter into the tube 205 through the side opening 1105 into the tube passageway having an inner surface with additional protrusions as taught by the previous exemplary embodiment. The protrusions 1115 disposed on the inner surface of the side opening 1105 may prevent the thread 1120 from moving or slipping out of the tube 205. Such an exemplary embodiment may eliminate the need for a guide as previously discussed that pulls the suture through the tube. Instead, in this exemplary embodiment shown in FIG. 11A, the suture may be pushed or pressed into the longitudinal side of the tube. As seen in FIG. 11B, once the suture is inserted into the tube, the protrusions within the passageway of the tube may operate similarly to the previously described protrusions illustrated in FIGS. 2A-9C, and thus a further explanation thereof will be omitted. Additionally, as seen in FIGS. 11A-11B a handle 1110 may be further provided on an exterior surface of the suture lock. The handle 1110 may be formed in any shape that allows a medical instrument, such as forceps, or allows medical personnel to grasp the suture lock to move the suture lock toward a surgical site, to facilitate the insertion of the suture threads into the tube, or the like.

As discussed above, the knotting tube of the claimed disclosure is capable of reducing errors occurring during the process of suture tying while reducing training time for medical staff. The particular design of the knotting tube eliminates the need for complex techniques and external devices. The tube may also be made of a material to encourage cell growth over the tube itself to absorb the tube. Accordingly, the knotting tube of the claimed disclosure provides a single integrated device capable of simplifying the process of suture tying for medical procedures.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A suture lock, comprising:
a tube having a passageway formed therethrough;
a plurality of protrusions extending from at least a portion of an inner surface of the tube to pierce a suture,
wherein the tube receives both ends of the suture through the passageway and is moved to abut a surgical site, and
wherein the protrusions are deformable in one direction to lock the suture and maintain the tube abutting the surgical site.

2. The suture lock of claim 1, wherein the protrusions are formed at varied lengths and are formed at an angle less than 90 degrees.

3. The suture lock of claim 1, wherein the protrusions are formed in various shapes including a barb, valve, thorn, and hook.

4. The suture lock of claim 1, wherein a longitudinal side of the tube is open to receive the suture through a side opening.

5. The suture lock of claim 4, further comprising:
a handle disposed at an opposite side of the side opening.

6. The suture of claim 1, wherein the inner surface of the tube includes a rough topography.

7. The suture of claim 1, wherein two tubes are disposed adjacently to receive multiple sutures.

8. The suture of claim 1, further comprising a guide configured to pull both ends of the suture through the tube passageway.

9. The suture lock of claim 1, wherein the suture lock is formed in a bead shape having protrusions formed on an inner surface thereof to pierce the suture.

10. The suture lock of claim 1, wherein the tube has a cross-sectional shape selected from any one of the group consisting of: circular, rectangular, and triangular.

11. The suture lock of claim 1, wherein the tube is made of a biomaterial.

12. A method of locking a suture against tissue, comprising:
pulling the suture through the tissue by piercing a needle engaged with the suture through the tissue;
passing ends of the suture through a tube of a suture lock;
pushing the suture lock against the tissue; and
engaging the suture in the tube with protrusions extending from a least a portion of an inner surface of the tube to pierce the suture and lock the suture against the tissue.

13. The method of claim 12, wherein the protrusions are formed at varied lengths and are formed at an angle less than 90 degrees.

14. The method of claim 12, wherein the protrusions are formed in various shapes including a barb, valve, thorn, and hook.

15. The method of claim 12, further comprising:
passing the ends of the suture through a side opening formed at a longitudinal side of the tube.

16. The method of claim 12, wherein the inner surface of the tube includes a rough topography to engage with the suture to lock the suture against the tissue.

17. The method of claim 12, wherein the suture lock is formed in a bead shape having protrusions formed on an inner surface thereof to pierce the suture.

18. The method of claim 12, wherein the tube has a cross-sectional shape selected from any one of the group consisting of: circular, rectangular, and triangular.

19. The method of claim 12, wherein the tube is made of a biomaterial.
